(19) United States
(12) Patent Application Publication
(10) Pub. No.: US 2020/0353231 A1
(43) Pub. Date: Nov. 12, 2020

(54) IMPLANTABLE CATHETER ASSEMBLIES

(71) Applicant: MICROBOT MEDICAL LTD., Caesarea (IL)

(72) Inventors: Simon SHARON, Maayan Zvi (IL); Or SAMOOCHA, Bustan Hagalil (IL); Idan BOADER, Karmiel (IL)

(21) Appl. No.: 16/965,986

(22) PCT Filed: Jan. 31, 2019
(86) PCT No.: PCT/IL2019/050121

§ 371 Date: Jul. 29, 2020

Related U.S. Application Data
(60) Provisional application No. 62/625,928, filed on Feb. 2, 2018.
(60) Provisional application No. 62/767,613, filed on Nov. 15, 2018.
(60) Provisional application No. 62/784,729, filed on Dec. 25, 2018.

(51) Int. Cl. A61M 27/00; A61M 25/00; B08B 9/051

(52) U.S. Cl. CPC A61M 27/006; A61M 25/007; B08B 9/051; B08B 2209/04; A61M 2205/0272; A61M 2205/8206; A61M 2025/0019

(57) ABSTRACT

Disclosed is an implantable catheter for fluid passage, including a catheter tube, a catheter tip member including one or more apertures and an open proximal end, and a cleaning unit at least partially positioned within the catheter tip member. The catheter tip member is connected at the proximal end thereof to the catheter tube, thereby fluidly coupling the catheter tube to an exterior of the catheter. The cleaning unit is configured for movement within the catheter tip member such as to mechanically prevent, mitigate, and/or remove blockage in the one or more apertures when the catheter tip member is implanted in a body cavity. The catheter tip member further includes a stopper configured to be engaged by a tip portion of a mandrel, such as to prevent the mandrel from reaching and/or damaging the cleaning unit when using the mandrel to guide the catheter tip member into the body cavity.

### TECHNICAL FIELD

The present disclosure relates generally to implantable catheters for fluid delivery, drainage, and/or passage.

### BACKGROUND

Shunts are often used as internal medical devices to drain aberrant fluids from different organs. FIG. 1A schematically depicts a prior art cerebral shunt 15 for draining cerebrospinal fluid (CSF) implanted in an infant patient 25. Shunt 15 includes a ventricular catheter 35, a drain tube 37, and a valve 39 regulating the flow of fluid from ventricular catheter 35 to drain tube 37. Ventricular catheter 35 is implanted in a brain ventricle (not indicated). FIG. 1B is a close-up view of ventricular catheter 35. A catheter head 41 of ventricular catheter 35 includes a plurality of apertures 47 and 49, along its length; the apertures often having different sizes and different spacings, such that CSF accumulated around ventricular catheter 35 drains through the apertures into drain tube 37, and away from the brain ventricle. The excess CSF is generally drained into a body cavity such as the abdomen. Ventricular catheter 35 may have length calibrations imprinted thereon, so that the surgeon can estimate how far ventricular catheter 35 has been inserted into the cranial cavity. Drain tube 37 is generally implanted just beneath the skin, with access to the cranial region to be drained, and into the abdominal cavity, being achieved by means of small incisions 55 in the meninges and the peritoneum respectively. To allow the patient to grow into adulthood without having to replace the shunt, an end section 61 of drain tube 37 may be bundled up in the abdominal cavity, so that it can unravel as the patient grows.

Such prior art simple shunts, as described above, generally have two major problems: (i) the inlet apertures might get clogged, and (ii) the ventricular catheter might become contaminated and thereby potentially cause an infection. When the ventricular catheter becomes clogged (e.g. due to clogging of the inlet apertures), an attempt to remove it from the body by surgery should be made. In cases where it is impossible to remove, another ventricular catheter may be placed in parallel to the malfunctioning one. When the ventricular catheter is contaminated it must be removed from the body by surgery. Surgeries of this kind are often high-risk procedures.

The simple prior art shunts depicted in FIGS. 1A and 1B have a significant drawback in that after some period of time inside the human body, living tissue growth may result in blockage of the apertures by the tissue. This tissue is generally the main cause of shunt blockage. When trying to withdraw the shunt by surgery, the ingrown tissue may tear, causing intraventricular bleeding, which might be life threatening.

### SUMMARY

Aspects of the disclosure, according to some embodiments thereof, relate to implantable catheters, shunts, delivery ports, and the like, used in fluid delivery, drainage, and/or passage, such as ventricular catheters for draining cerebrospinal fluid (CSF), external ventricular drainage (EVD) catheters, urinary catheters, and delivery ports (e.g. for chemotherapy). More specifically, but not exclusively, aspects of the disclosure, according to some embodiments thereof, relate to catheters/shunts/delivery ports wherein a tip member of the catheter/shunt/delivery port includes apertures for fluid passage and further includes a cleaning unit configured to prevent the apertures from becoming blocked, e.g. by tissue growth (such as choroid plexus in the brain ventricles), cells, minerals, and/or coagulated blood.

Such cleaning units tend to be fragile and, during implantation of the catheter/shunt/delivery port, may be damaged by a mandrel used to guide the catheter/shunt/delivery port through bodily passages to their target location in the body. To prevent such damage from occurring, advantageously, the catheter/shunt/delivery port may include a stopper configured to be mechanically engaged by a tip portion of the mandrel during the implantation.

According to some embodiments, the cleaning unit includes two sets of arms extending on opposite sides of a central shaft of the cleaning unit and into the apertures in the walls of the tip member. Advantageously, the stopper and the tip portion may include complementary key patterns configured to allow a surgeon to rotationally orient the catheter tip member within a body cavity such as a ventricle (during implantation thereof) to a preferred orientation wherein, e.g. when the subject (e.g. patient) is standing or sitting upright, the two sets of arms are level, that is, equally or substantially equally supported by the apertures. This allows activating the cleaning unit in a known orientation, and, in particular, in a favorable orientation, i.e. when the two sets of arms are level. Thus, for example, in the case that the cleaning unit is intended to be activated when the subject is standing, the cleaning unit will be oriented differently during the implantation thereof, than in the case that the cleaning unit is intended to be activated when the subject is lying down/on their side.

More generally, the skilled person will appreciate that the scope of the disclosure, according to some embodiments, covers any medical implant (e.g. a tubular implant, a distally located structure) housing delicate components and including a stopper (such as the stoppers disclosed herein) which is guided to a target site within the body using a mandrel configured to engage the stopper (such as the mandrels disclosed herein), thereby preventing damage to the delicate components housed in the implant. The skilled person will further appreciate that the scope of the disclosure, according to some embodiments, covers any medical implant housing delicate components and including a keyed stopper (i.e. a stopper including a key pattern), such as the keyed stoppers disclosed herein, which has to be oriented within a target site in the body using a corresponding keyed mandrel, such as the keyed mandrels disclosed herein, thereby preventing damage to the delicate components housed in the implant. The delicate components may, for example, be mechanical, electronic, electromechanical, magnetic, and/or electromagnetic. In particular, the skilled person will appreciate that the scope of the disclosure, according to some embodiments, also covers implants which are not used for fluid passage and/or which do not include internal cleaning units. For example, medical implants, which are guided to a target site in the body using a mandrel, and which include (e.g. house) delicate components, such as flow sensors, pressure sensors, pH sensors, and the like.

Thus, according to an aspect of some embodiments, there is provided an implantable catheter for fluid passage, including:
A catheter tube.
An elongated and hollow catheter tip member including one or more apertures and an open proximal end. The catheter tip member is connected at the proximal end thereof to the catheter tube, such as to fluidly couple (via the one or more apertures) the catheter tube to an exterior (the outside) of the catheter.
A cleaning unit at least partially positioned within the tip member distal section.

The cleaning unit is configured for movement within the catheter tip member such as to mechanically prevent, mitigate, and/or remove blockage of at least one of the one or more apertures when the catheter tip member is implanted in a body cavity. The catheter tip member includes a stopper configured to be engaged by a tip portion of a mandrel, such as to prevent the mandrel from at least one of reaching and damaging the cleaning unit when using the mandrel to guide the catheter tip member into the body cavity.

According to some embodiments, the catheter tip member includes a tip member proximal section and a tip member distal section. The tip member proximal section includes the stopper and the tip member distal section includes the one or more apertures. The cleaning unit is at least partially housed within the tip member proximal section.

According to some embodiments, the catheter is a ventricular catheter for draining fluids. The fluids may include cerebrospinal fluid (CSF), and the body cavity may include a brain ventricle.

According to some embodiments, the stopper includes a first geometrical feature projecting from an inner surface of the tip member proximal section.

According to some embodiments, the stopper includes a first key pattern and the tip portion of the mandrel includes a second key pattern complementary to the first key pattern. The first and second key patterns may be configured to interlock, upon engaging of the stopper by the tip portion of the mandrel, such that a rotation of the mandrel induces an equal rotation of the catheter tip member.

According to some embodiments, the first key pattern may be configured as male and the second key pattern may be configured as female, or the first key pattern may be configured as female and the second key pattern may be configured as male.

According to some embodiments, the tip portion of the mandrel includes a second geometrical feature radially projecting relative to a main body of the mandrel. The second geometrical feature may be configured to engage the first geometrical feature.

According to some embodiments, the tip portion of the mandrel includes a band or a flange therearound configured to engage the stopper.

According to some embodiments, the first geometrical feature may include a flange extending along a circumference of the inner surface, or the first geometrical feature may include a narrowed segment of a lumen defined by the inner surface.

According to some embodiments, the first geometrical feature includes at least two spaced-apart ridges along a circumference of the inner surface.

According to some embodiments, the first geometrical feature includes a first key pattern and the second geometrical feature includes a second key pattern complementary to the first key pattern. The key patterns may be configured to interlock upon engaging of the stopper by the tip portion of the mandrel, such that a rotation of the mandrel induces an equal rotation of the catheter tip member.

According to some embodiments, the first key pattern comprises at least one slot in the first geometrical feature (e.g. the narrowed segment) and the second key pattern includes at least one projection (e.g. tooth) extending distally from a distal end of the second geometrical feature (e.g. the band). The at least one slot is complementary to the at least one projection.

According to some embodiments, the first key pattern includes at least one projection (e.g. tooth) extending proximally from a proximal end of the first geometrical feature (e.g. narrowed segment) and the second key pattern includes at least one slot in the second geometrical feature (e.g. the band). The at least one slot is complementary to the at least one projection.

According to some embodiments, the cleaning unit includes an elongated shaft including one or more arms configured to project into the one or more apertures and to move therein.

According to some embodiments, the cleaning unit is configured to allow vibration thereof. The movement of the arms within the one or more apertures may be induced by the vibration of the cleaning unit.

According to some embodiments, the vibration of the cleaning unit includes at least one of reciprocal motion thereof along the catheter tip member and tilting of the cleaning unit.

According to some embodiments, the one or more apertures include at least two apertures on opposite walls of the tip member distal portion.

According to some embodiments, the one or more apertures include a plurality of apertures arranged in two longitudinal, or substantially longitudinal, rows on opposite walls of the tip member distal portion.

According to some embodiments, the arms of the cleaning unit extend into the apertures such as to suspend the cleaning unit within the catheter tip member.

According to some embodiments, the implantable catheter further includes a vibration generator configured to induce the movement of the cleaning unit. The vibration generator is connected to an electrical line which is configured to couple to a power supply unit for powering the vibration generator.

According to some embodiments, the electrical line proximally extends along at least a part of the catheter tube from the distal end thereof.

According to some embodiments, the electrical line is winded along said at least a part of the catheter tube.

According to some embodiments, the electrical line is embedded within walls of the catheter tube.

According to some embodiments, the vibration generator is at least partially housed within the tip member proximal section.

According to some embodiments, the cleaning unit includes the vibration generator or a part thereof.

According to some embodiments, the catheter tube includes a port into which the electrical line extends. The port is configured to electrically couple to the power supply unit.

According to some embodiments, the power supply unit is implantable.

According to some embodiments, the power supply unit includes a second coil of conducting wire.

According to some embodiments, the catheter system further includes a power supply unit, as described above, and a flexible extension associating the power supply unit with the port and wherethrough the electrical line extends.

According to some embodiments, the cleaning unit includes a metallic member (e.g. a casing or bar) including at least one of a magnetic and magnetizable material. The metallic member may be attached to a proximal end of the shaft. The tip member proximal section may further include an electrically conducting coil such that the metallic member and the electrically conducting coil are configured as an electromagnet constituting, or included in, the vibration generator.

According to some embodiments, the catheter tip member is integrally formed.

According to some embodiments, the tip member distal section is made of a material including at least one of a corrosion resistant material, non-toxic material, and non-magnetic material.

According to some embodiments, the tip member proximal section is made of a material including at least one of rubber and plastic.

According to some embodiments, the tip member distal section is made of a material including titanium.

According to some embodiments, the tip member proximal section may be made of a material including titanium and a proximal portion of the tip member proximal section may be covered by silicone.

According to some embodiments, wherein the tip member proximal section includes the metallic member and the coil described above, the coil may be winded about a wall of the tip member proximal section and may be coated with an electrically insulating material.

According to some embodiments, the electrically insulating material includes silicone.

According to some embodiments, a proximal end of the catheter tube includes, or is configured to be connected to, a one-way valve, which is configured to allow fluid flow therethrough only in the proximal direction therefrom.

According to an aspect of some embodiments, there is provided a kit for fluid passage in a body cavity. The kit includes an implantable catheter, as described above, and a mandrel, as described above.

According to some embodiments, the mandrel is pre-installed in the implantable catheter such that the tip portion of the mandrel engages the stopper in the catheter tip member.

According to some embodiments, wherein the catheter tip member and the mandrel include complementary key patterns as described above, a proximal portion of the mandrel may proximally extend outside the catheter tube, and the mandrel may include an orientation indicator.

According to some embodiments, the proximal portion of the mandrel is coiled and constitutes at least part of the orientation indicator.

According to some embodiments, the orientation indicator includes at least one of a notch and a color marking on the proximal portion of the mandrel.

According to some embodiments, a torsional stiffness of the mandrel is such as to allow the mandrel to rotate the catheter tip member without, or substantially without, the mandrel being simultaneously twisted about a longitudinal axis thereof.

According to some embodiments, a proximal section of the catheter tube is attached to the proximal portion of the mandrel, thereby facilitating maintaining the engaging of the stopper by the tip portion of the mandrel when guiding the catheter tip member into the body cavity.

According to some embodiments, the proximal section of the catheter tube is detachable from the rest of the catheter tube, thereby facilitating removing the mandrel from the implantable catheter.

According to some embodiments, the proximal section of the catheter tube is joined to the rest of the catheter tube by a thin segment of weakened material, thereby facilitating detaching the proximal section of the catheter tube.

According to some embodiments, wherein the catheter tube includes a port as described above, the kit further includes a power supply unit as described above.

According to some embodiments, the kit further includes a flexible extension configured to electrically couple the port to the power supply unit.

According to some embodiments, the blockage may result from tissue entering into at least one of the one or more apertures when the catheter tip member is implanted in the body cavity.

According to an aspect of some embodiments, there is provided a device for mitigating obstructions in a medical implant. The device includes:
A tubular conduit having a plurality of fluid openings and configured for implantation within an anatomical body for at least one of fluid delivery, fluid drainage, and fluid passage.
An elongated lumen extending from the tubular conduit and configured to be fluidly-communicable with the tubular conduit.
At least one movable element located at least partially within the tubular conduit and being configured to move within the tubular conduit to impede obstruction of at least one of the plurality of fluid openings.
An elongated shaft configured to be removably inserted into the elongated lumen through an open proximal end of the elongated lumen.
A stopper located within at least one of a proximal end of the tubular conduit and a distal end of the elongated lumen, the stopper being configured to prevent the elongated shaft from reaching the at least one moveable element.

According to some embodiments, the stopper is additionally configured to allow fluid flow along the elongated lumen.

According to some embodiments, the elongated shaft is a mandrel configured to enable the implantation of the tubular conduit within the anatomical body. According to some embodiments, a distal end of the mandrel is keyed to be received in a keyed opening in the stopper.

According to some embodiments, the at least one moveable element includes a single element with protrusions configured to mitigate obstruction of at least one of the fluid openings.

According to some embodiments, the at least one moveable element includes a plurality of cleaning units, each being associated with a respective fluid opening.

According to some embodiments, at least one of the fluid openings is located in a fluid receiving tip of the tubular conduit, wherein the elongated lumen extends proximally from a proximal end of the fluid receiving tip. According to some embodiments, the device further includes a keyed connector located near the proximal end of the fluid receiving tip, in one of the fluid receiving tip and the elongated lumen. According to some embodiments, the removable elongated shaft includes a keyed distal end configured to engage with the keyed connector. According to some embodiments, the keyed distal end of the removable elongated shaft includes a male configuration, and the keyed connector includes a female receiver.

According to some embodiments, the device further includes a guide associated with a proximal location on the removable elongated shaft and configured to assist a medical professional in rotationally orienting the fluid receiving tip upon its implantation within the anatomical body, based on a rotational orientation of the guide. According to some embodiments, the guide includes a non-circular shape to advise the medical professional of the rotational orientation of the fluid receiving tip within the anatomical body.

According to some embodiments, the guide includes at least one radial marking to advise the medical professional of the rotational orientation of the fluid receiving tip within the anatomical body.

According to some embodiments, the fluid receiving tip, the elongated lumen, and the removable elongated shaft are configured to traverse a brain of the patient while the shaft is within the elongated lumen, to facilitate placement and rotational orientation of the fluid receiving tip within the brain.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1A schematically depicts a prior art cerebral shunt for draining cerebrospinal fluid from a ventricle in a brain of subject;

FIG. 1B schematically depicts a (prior art) ventricular catheter assembly of the cerebral shunt of FIG. 1A;

FIG. 2A is a schematic perspective view of a ventricular catheter assembly including a ventricular catheter, according to some embodiments;

FIG. 2B is a schematic perspective view of a catheter tip member of the ventricular catheter of FIG. 2A, according to some embodiments;

FIG. 3A is a schematic, top, cross-sectional view of the ventricular catheter of FIG. 2A and a mandrel inserted therein, such as to engage the catheter tip member, according to some embodiments;

FIG. 3B is a schematic, side, cross-sectional view of the ventricular catheter and the mandrel of FIG. 3A, according to some embodiments;

FIG. 4 is a schematic, perspective view of a cleaning unit and a vibration generator of the ventricular catheter of FIG. 3A, according to some embodiments;

FIG. 5A is a schematic, side, cross-sectional view of the mandrel and a proximal section of the catheter tip member of FIG. 3A, according to some embodiments;

FIG. 5B is a schematic, perspective, exploded view of the mandrel and the proximal section of the catheter tip member of FIG. 3A, according to some embodiments;

FIG. 5C is a schematic, front view of the mandrel and the proximal section of the catheter tip member of FIG. 3A, according to some embodiments;

FIG. 6A is a schematic, side, cross-sectional view of a proximal section of a catheter tip member of a ventricular catheter and a mandrel engaging the catheter tip member, such as to interlock with the catheter tip member, according to some embodiments;

FIG. 6B is a schematic, perspective, exploded view of the mandrel and the proximal section of the catheter tip member of FIG. 6A, according to some embodiments;

FIG. 6C is a schematic, front view of the mandrel and proximal section of the catheter tip member of FIG. 6A, according to some embodiments;

FIG. 6D is a schematic, perspective view of the mandrel and the proximal section of the catheter tip member of FIG. 6A, according to some embodiments;

FIG. 7A is a top, cross-sectional view of the catheter tip member of FIG. 6A with the catheter tip member being oriented such that opposite arms of a cleaning unit, housed therein, are level, according to some embodiments; and

FIG. 7B is a top, cross-sectional view of the catheter tip member of FIG. 6A with the catheter tip member and the cleaning unit being oriented at 90° relative to the orientation thereof in FIG. 7A, according to some embodiments.

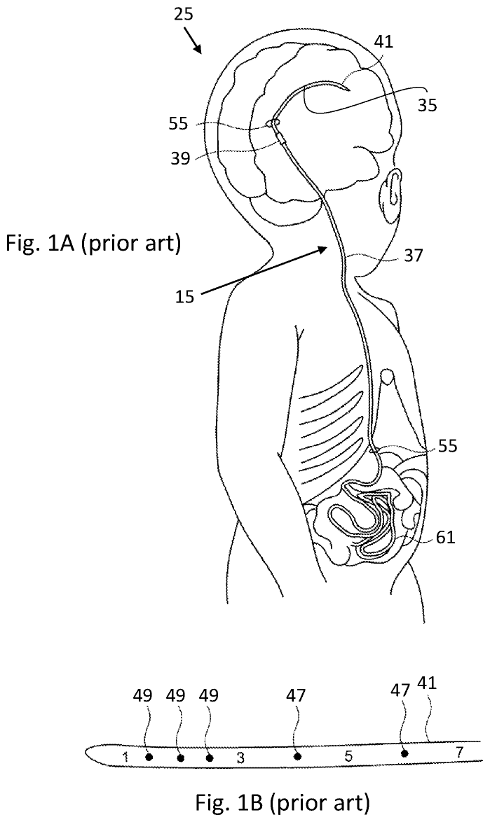

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application the expression “at least one of A and B”, (e.g. wherein A and B are elements, method steps, claim limitations, etc.) is equivalent to “only A, only B, or both A and B”. In particular, the expressions “at least one of A and B”, “at least one of A or B”, “one or more of A and B”, and “one or more of A or B” are interchangeable.

In the description and claims of the application, the words “include” and “have”, and forms thereof, are not limited to members in a list with which the words may be associated.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

For ease of description, in some of the figures a three-dimensional cartesian coordinate system (with orthogonal axes x, y, and z) is introduced. It is noted that the orientation of the coordinate system relative to a depicted object may vary from one figure to another. Further, the symbol ⊙ is used in the figures to represent an axis pointing “out of the page”, and the symbol ⊗ is used in the figures to represent an axis pointing “into the page”.

As used herein, according to some embodiments, a “proximal” end/section/portion/tip of an element/component/device may refer to a part of the element/component/device that is closer to a surgeon or a medical practitioner (e.g. during implantation of the device) as compared to at least one other part of the element/component/device. Similarly, according to some embodiments, a “distal” end/section/portion/tip of an element/component/device may refer to a part of the element/component/device that is further from a surgeon or a medical practitioner (e.g. during implantation of the device) as compared to at least one other part of the element/component/device. According to some embodiments, a “distal” end/section/portion/tip of an element/component/device may refer to a part of the element/component/device that is closer to a diagnosis or treatment site in the body of a patient as compared to at least one other part of the element/component/device.

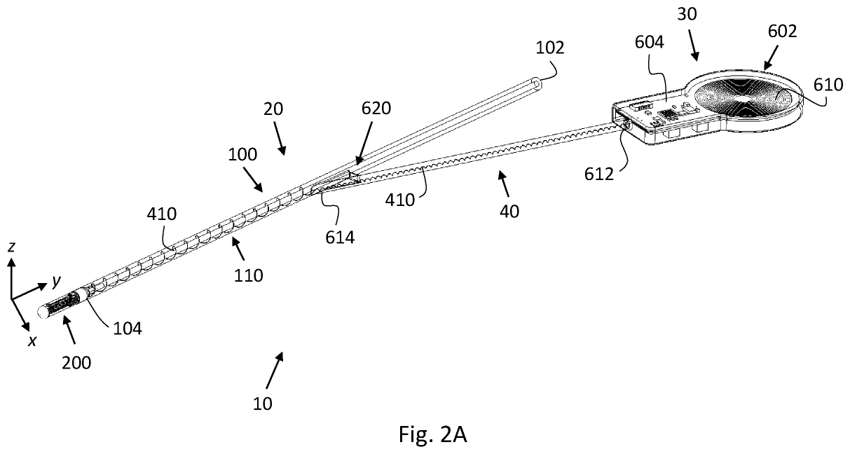

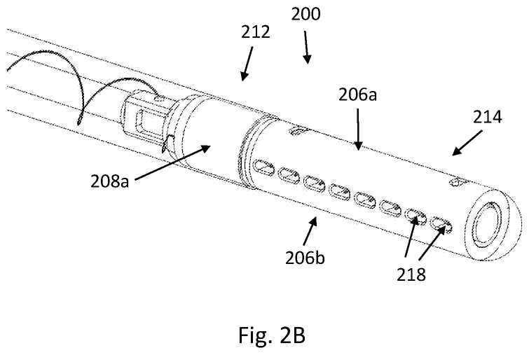

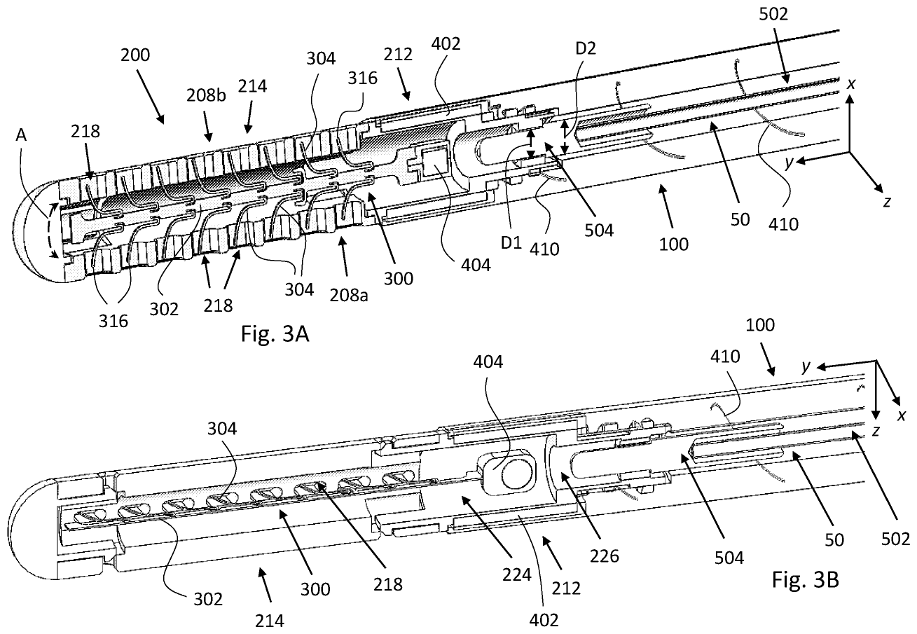

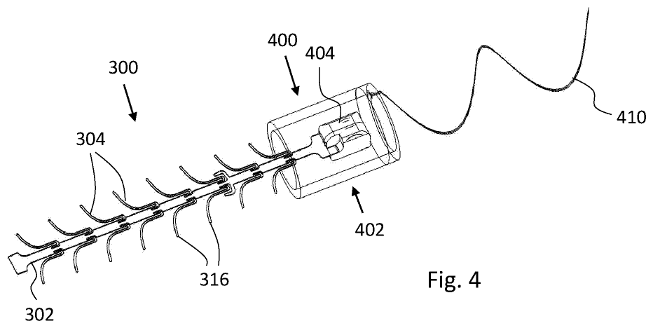

FIG. 2A is a schematic, perspective view of a ventricular catheter assembly 10, according to some embodiments. Catheter assembly 10 includes a ventricular catheter 20, a power supply unit 30, and a flexible extension 40 (e.g., a tube/cable) associating ventricular catheter 20 and power supply unit 30, as elaborated on below. Catheter 20 includes an elongated catheter tube 100, a catheter tip member 200, a cleaning unit 300, and a vibration generator 400 (shown in FIGS. 3A-4). According to some embodiments, both power supply unit 30 and flexible extension 40 are also implantable. According to some such embodiments, power supply unit 30 is implantable beneath the skin but outside the skull while flexible extension 40 is implantable (under the skull but) outside the ventricle. According to some other such embodiments, both power supply unit 30 and flexible extension 40 are implantable beneath the skin but outside the skull. It is noted that, in accordance with some embodiments, flexible extension 40 and/or power supply unit 30, may be detachable and may be connected to ventricular catheter 20 (e.g., via a port, not shown) after the implantation of ventricular catheter 20.

FIG. 2B is a schematic, perspective view of catheter tip member 200, according to some embodiments.

Making reference also to FIGS. 3A and 3B, FIG. 3A is a schematic, top, cross-sectional view of catheter 20 with a mandrel 50 inserted thereinto, according to some embodiments. FIG. 3B is a schematic, side, cross-sectional view of catheter 20 and mandrel 50, according to some embodiments. That is, the view in FIG. 3B is taken at 90° relative to FIG. 3A. Mandrel 50 is shown engaging catheter tip member 200, as elaborated on below.

Catheter tube 100 extends from a tube proximal end 102 (shown in FIG. 2A) to a tube distal end 104. Tube proximal end 102 may be configured to be connected to a valve (not shown), such as valve 39, as elaborated on below. Tube distal end 104 is joined to catheter tip member 200, as elaborated on below.

Catheter tip member 200 is hollow (as seen in FIGS. 3A and 3B) and is open on a tip member proximal end 202 (i.e. the proximal end of catheter tip member 200). According to some embodiments, catheter tip member 200 may be shaped as a short tube. Catheter tip member 200 includes a top surface 206a, a bottom surface 206b, a first side surface 208a adjacent to both top surface 206a and bottom surface 206b, and a second side surface 208b (shown in FIG. 3A) opposite to first side surface 208a.

Catheter tip member 200 further includes a tip member proximal section 212 (i.e. a proximal section of catheter tip member 200; the proximal section including tip member proximal end 202) and a tip member distal section 214 (i.e. a distal section of catheter tip member 200). Tip member proximal section 212 and tip member distal section 214 are joined.

Tip member distal section 214 includes apertures 218 (not all of which are numbered) wherethrough fluids can (i) enter catheter tip member 200 from outside thereof, when the catheter is utilized for fluid drainage/passage, and (ii) exit catheter tip member 200 to the outside thereof, when the catheter is utilized for fluid delivery/passage. Tip member proximal end 202 is connected to tube distal end 104, thereby fluidly connecting apertures 218 to catheter tube 100 and allowing to (i) expel, via catheter tube 100, fluids (e.g. CSF from a brain ventricle) drained through apertures 218, or (ii) deliver, via catheter tube 100 and apertures 218, fluids (e.g. medication) to a target site/location within a patient's body. According to some embodiments, and as depicted in the figures, apertures 218 are arranged in two rows of apertures: a first row and a second row (not numbered). The two rows may extend along the length of tip member distal section 214 on opposite sides thereof, as depicted, for example, in FIG. 3A, i.e. on first side surface 208a and second side surface 208b, respectively. According to some embodiments, apertures 218 may be round. According to some embodiments, apertures 218 may be elongated, e.g. in the form of slots.

FIG. 4 is a schematic, perspective view of cleaning unit 300 and vibration generator 400, according to some embodiments. Cleaning unit 300 (depicted also in FIGS. 3A and 3B) is at least partially housed within tip member distal section 214. According to some embodiments, cleaning unit 300 includes a central shaft 302 and arms 304 (not all of which are numbered) extending from shaft 302, as disclosed, for example, in U.S. Pat. No. 9,393,389, titled “Self Cleaning Shunt”, to Samoocha et al., which is incorporated herein by reference in its entirety. According to some embodiments, arms 304 include two sets of arms: a first set and a second set (not numbered). According to some embodiments, shaft 302 and arms 304 span or substantially span a plane (e.g. shaft 302 and arms 304 lie or substantially lie in parallel to the xy-plane in FIG. 3A).

According to some embodiments, shaft 302 is longitudinally or substantially longitudinally disposed within catheter tip member 200. That is, shaft 302 may be disposed or substantially disposed in parallel to the y-axis (at least when cleaning unit 300 is not vibrating). According to some embodiments, arms 304 may be capable of projecting from shaft 302 such that tips 316 of arms 304 reach into apertures 218. According to some embodiments, arms in the first set are positioned such as to allow each of the arms to extend into a respective aperture from the first row of apertures (e.g. the distances between adjacent arms in the first set equal or substantially equal the distances between adjacent apertures in the first row), and arms in the second set are positioned such as to allow each of the arms to extend into a respective aperture from the second row of apertures.

According to some embodiments, shaft 302 may be configured for motion/oscillation along and/or about a longitudinal axis of catheter tip member 200. (The longitudinal axis runs parallel to the y-axis.) Arms 304 may be configured for movement (e.g. of tips 316) within apertures 218 such as to prevent tissue from entering/blocking apertures 218 and/or to remove/clear/push-out tissue which has entered/blocked one or more of apertures 218 (when catheter 20 is implanted in a ventricle, for example). According to some embodiments, shaft 302 is configured for movement (e.g. vibration) such as to induce movement of arms 304/tips 316 within apertures 218. The movement of each of arms 304/tips 316 may be such as to range over all the area of the respective aperture, so as to ensure that tissue does not penetrate into the aperture. In particular, shaft 302 may be configured for tilting motion (as indicated by a curved double-headed arrow A in FIG. 3A), so as to effect radial movement of arms 304 within apertures 218, wherein the depth of penetration of an arm into a respective aperture alternately increases and decreases. According to some embodiments, the length(s) of arms 304 is determined according to the thickness of the walls (not numbered) of tip member distal section 214 such that tips 316 do not (e.g. cannot) protrude out of tip member proximal section 212, particularly when cleaning unit 300 vibrates.

According to some embodiments, arms from the first set and the second set extend into apertures from the first row and the second row, respectively, thereby suspending cleaning unit 300 within catheter tip member 200 (e.g. tips 316 remain within apertures 218, in particular, when cleaning unit 300 is activated). That is, apertures 218 support cleaning unit 300 within catheter tip member 200. Further, movement of cleaning unit 300 within catheter tip member 200 is restricted, since the movement of tips 316 is restricted by the dimensions of apertures 218.

Additionally/alternatively, according to some embodiments, cleaning unit 300 may be supported/partially supported by a pin (not indicated) oriented at substantially right angles to shaft 302 (e.g. in parallel to the z-axis) and extending through a hole (not shown) in shaft 302. The pin may function as a pivot about which shaft 302 oscillates when cleaning unit 300 is activated.

Vibration generator 400 (e.g. an electromagnet or an electric or electromechanical motor) is configured to induce movement/vibration of shaft 302 (and arms 304). According to some embodiments, vibration generator 400 is mechanically coupled to cleaning unit 300. According to some embodiments, vibration generator 400 forms part of cleaning unit 300.

According to some embodiments, and as depicted in the figures, some components of vibration generator 400 are included in catheter tip member 200 and other components of vibration generator 400 are included in cleaning unit 300. According to some embodiments, vibration generator 400 is an electromagnet including a coil 402 (of an electrically conducting wire) and a metallic casing 404 (e.g. a metal cylinder, shown also in FIGS. 3A and 3B). Metallic casing 404 may be or include a magnet (e.g. a neodymium magnet) and/or a magnetizable material, and may be housed in a chamber 224 inside tip member proximal portion 212. According to some embodiments, the magnet is enclosed in a corrosion-resistant metallic (e.g. titanium) casing and/or is coated with a biocompatible material. Coil 402 may be winded (wound) about a wall (not numbered, e.g. externally on the wall) of chamber 224. According to some embodiments, coil 402 is coated by an electrically-insulating material, e.g. a silicone coating or a parylene coating, or may be covered by a distal portion of catheter tube 100. Metallic casing 404 may be attached to a proximal end (not numbered) of shaft 302 such as to be at least partially disposed within coil 402. An electrical line 410 (e.g. two or more electrical wires housed within a cable; shown also in FIG. 2A) is connected to coil 402 and is configured to supply electrical current to power vibration generator 400, as elaborated on below. More specifically, electrical line 410 extends from coil 402 in the proximal direction along at least a tube distal section 110 (i.e. a distal section of catheter tube 100) and is electrically coupled/connected to a power supply component, as elaborated on below. According to some embodiments, at least along tube distal section 110, electrical line 410 is embedded within the walls of catheter tube 100. According to some such embodiments, at least along tube distal section 110, electrical line 410 is winded within the walls thereof.

According to some embodiments, not shown in the figures, electrical line 410 constitutes or includes conductive tracks (e.g. copper tracks) on a strip of a printed circuit board (PCB). The PCB strip may be housed within walls (not numbered) of catheter tube 100.

According to some embodiments, not depicted in the figures, vibration generator 400 is implanted in the head outside the ventricle or even the skull, and is mechanically coupled to cleaning unit 300 via mechanical infrastructure which is configured to impart motion of vibration generator 400 to cleaning unit 300 and which extends at least through tube distal section 110. According to some embodiments, the mechanical infrastructure may include, for example, a resilient rod/wire (the wire may be similar, or mechanically similar, to a guidewire). Advantageously, according to some embodiments, all electric and electronic components, or at least all electric and electronic components involved in cleaning unit 300 maneuvering/motion, are located outside the skull or at least outside the ventricle.

According to some embodiments, not depicted in the figures, vibration generator 400 is or includes a piezoelectric motor, which is mechanically coupled to cleaning unit 300. According to some such embodiments, the piezoelectric motor is not housed in catheter tip member 200, instead being positioned more proximally. According to some embodiments, the piezoelectric motor is housed in a Y-junction (such as the Y-junction depicted in FIG. 2A and described below), which is located outside the skull or at least outside the ventricle, and is mechanically coupled to cleaning unit 300 via mechanical infrastructure as described above. According to some embodiments, the piezoelectric motor is housed in or near power supply unit 30, which is located outside the skull, and is mechanically coupled to cleaning unit 300 via mechanical infrastructure as described above (the infrastructure extending also through flexible extension 40). According to some alternative embodiments, the piezoelectric motor is housed in tip member proximal section 212 (which is located within the ventricle).

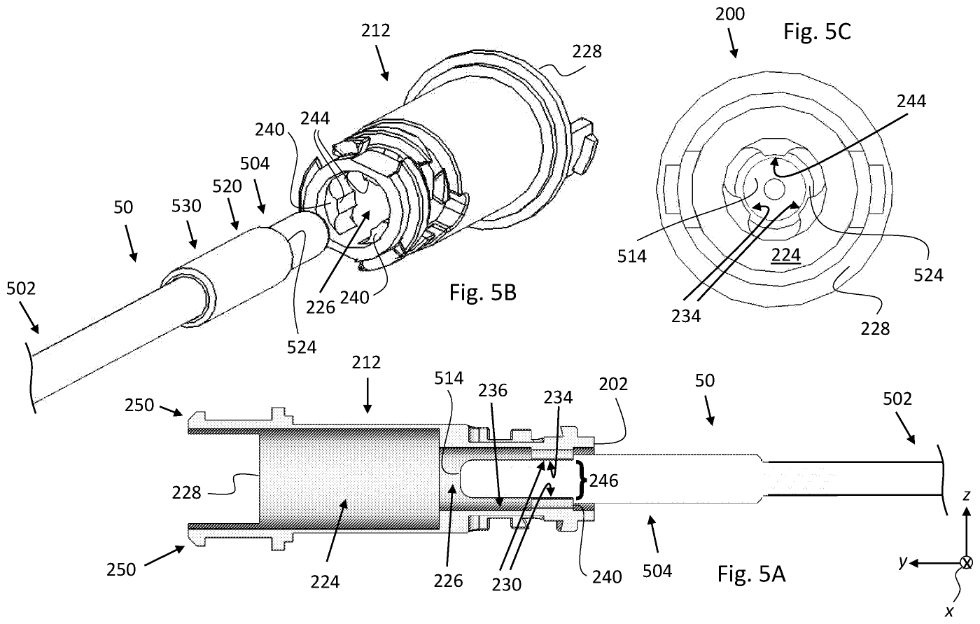

Making reference also to FIGS. 5A-5C, FIG. 5A is a schematic, side, cross-sectional view of tip member proximal section 212 and mandrel 50, which is shown engaging tip member proximal section 212, according to some embodiments. FIG. 5B is a schematic, perspective, exploded view of tip member proximal section 212 and mandrel 50. FIG. 5C is a schematic front view of tip member proximal section 212 and mandrel 50 (which is engaging tip member proximal section 212) taken facing the distal end of tip member proximal section 212.

Tip member proximal section 212 is configured to be engaged by mandrel 50 such as to facilitate inserting catheter 20 to its designated location within the body, e.g. to a brain ventricle. Mandrel 50 includes a mandrel main body 502 (e.g. a stiff wire or thin elongated shaft) and a mandrel tip portion 504 (i.e. a distal portion of mandrel 50). Mandrel main body 502 extends from a proximal end thereof (not indicated) to mandrel tip portion 504. Mandrel tip portion 504 terminates in a mandrel distal end 514 (the distal end of mandrel 50). Tip member proximal section 212 includes a lumen 226 extending longitudinally from tip member proximal end 202 to chamber 224. Lumen 226 is configured to receive mandrel tip portion 504. Also indicated is a distal end 228 of tip member proximal section 212.

According to some embodiments, tip member proximal section 212 includes a stopper 230 configured to be engaged by mandrel tip portion 504. According to some embodiments, stopper 230 includes/forms a first geometrical feature 234 projecting from a lumen wall 236 (inner surface) of lumen 226, and mandrel tip portion 504 includes a second geometrical feature 520 radially projecting (i.e. perpendicularly to the y-axis) relative to mandrel main body 502. That is, first geometrical feature 234 may be characterized by a first diameter (indicated in FIG. 3A by a double-headed arrow D1) which is smaller than the diameter of the rest of lumen 226, and second geometrical feature 520 may be characterized by a second diameter (indicated in FIG. 3A by a double-headed arrow D2) which is larger than the diameter of mandrel main body 502 (and larger than the first diameter characterizing first geometrical feature 234). Second geometrical feature 520 is configured to engage first geometrical feature 234 such as to facilitate guiding catheter 20 through ventricles. According to some embodiments, first geometrical feature 234 and second geometrical feature 520 may be complementary in the sense of defining surfaces which at least partially overlap (when mandrel tip portion 504 engages tip member proximal section 212), such as first surfaces 240 and a second surface 524 of first geometrical feature 234 and second geometrical feature 520, respectively.

According to some embodiments, first geometrical feature 234 and second geometrical feature 520 define mating surfaces.

According to some embodiments, mandrel tip portion 504 is integrally formed with mandrel main body 502. According to some embodiments, mandrel tip portion 504 includes a socket (not numbered) extending distally from the proximal end (not numbered) of mandrel tip portion 504, such as to allow mounting mandrel tip portion 504 on mandrel main body 502. According to some embodiments, mandrel tip portion 504 is welded or glued to mandrel main body 502.

According to some embodiments, first geometrical feature 234 constitutes a narrowed segment 246 of lumen 226 (as compared to the rest of lumen 226). According to some embodiments, first geometrical feature 234 constitutes a flange extending along the circumference of lumen wall 236. According to some embodiments, and as depicted in FIGS. 5A and 5B, second geometrical feature 520 is shaped as a band 530, or a flange, which is disposed around mandrel main body 502 (e.g. band 530 defines a region of mandrel tip portion 504 having a larger diameter than the diameter(s) of the rest of mandrel tip portion 504 and mandrel main body 502). According to some embodiments, wherein catheter 20 is configured to be implanted in a brain ventricle, narrowed segment 246 has a diameter of about 0.9 mm, and band 530 has a diameter of about 1.15 mm.

According to some embodiments, and as shown in FIGS. 5B and 5C, first geometrical feature 234 includes at least two spaced-apart ridges 244 (e.g. three in FIGS. 5B and 5C). Each of ridges 244 projects from lumen wall 236. The space between adjacent ridges (from ridges 244) may function to increase the fluid-flow cross-section via lumen 226, as compared to embodiments including a single annular ridge. The resultant increase in the fluid flow cross-section acts to minimize the influence of first geometrical feature 234 on the fluid flow through lumen 226.

It is noted that mandrel 50 does not directly or indirectly exert force on cleaning unit 300 and/or vibration generator 400 (specifically, when guiding catheter 20 through a ventricle, for example) except via the engaging of stopper 230 by mandrel tip portion 504 (so that by pushing stopper 230, the hull/frame of catheter tip member 200 is pushed, which in turn pushes cleaning unit 300 together with the hull/frame. In particular, stopper 230 prevents mandrel 50 from reaching/contacting cleaning unit 300 and/or vibration generator 400. This absence of exerted force ensures that cleaning unit 300 is not damaged by mandrel 50 during the insertion of catheter 20 into a ventricle.

As used herein, according to some embodiments, “indirectly exerted force” may refer to a mechanical force being exerted by a first element on a second element via one or more intermediary elements (e.g. a third element mechanically coupling the second element to the first element).

The skilled person will understand that the scope of the disclosure also covers embodiments wherein a distal section of catheter tube 100 includes a stopper (at or near tube distal end 104) in place of stopper 230 (i.e. in such embodiments, catheter tip member 200 does not include stopper 230). The stopper may be similar to stopper 230.

According to some embodiments, catheter tip member 200 is integrally formed. According to some embodiments, catheter tip member 200 includes, or is made of, a corrosion resistant, non-toxic, and/or non-magnetic material such as titanium.

According to some embodiments, tip member distal section 214 and tip member proximal section 212 are manufactured separately as two connectable parts (which, once assembled, are not detachable). According to some embodiments, and as depicted in FIG. 3B, tip member proximal section 212 and tip member distal section 214 are connected via a snap-fit mechanism. According to some such embodiments, and as depicted in the figures, tip member proximal section 212 constitutes the male, including one or more snaps 250 (indicated in FIG. 5A), and tip member distal section 214 constitutes the female. According to some embodiments, both tip member distal section 214 and tip member proximal section 212 include, or are made of, a corrosion resistant, non-toxic, and/or non-magnetic material, such as titanium. According to some embodiments, at least one of tip member distal section 214 and tip member proximal section 212 includes, or is made of, a polymeric material such as silicone. According to some embodiments, tip member proximal section 212 is made of titanium and covered with a silicone cover: over the coil 402 and proximally therefrom. The silicone cover may constitute a distal portion of catheter tube 100 or constitute a dedicated silicone coating.

In operation, once implanted in a patient, body fluids are drained/delivered/conducted via apertures 218. According to some embodiments, e.g. wherein catheter 20 is implanted in a brain ventricle and the body fluids are CSF, drained fluids may travel in the proximal direction from catheter tip member 200 into catheter tube 100, and therefrom via a drain tube, e.g. such as drain tube 37, into e.g. an abdominal cavity of the patient. More specifically, tube proximal end 102 may be connected to a valve regulating the flow of fluid into the drain tube. The valve may be a one-way valve thereby ensuring that fluid can only flow from catheter tube 100 to the drain tube and not in the opposite sense (or, only in the opposite sense, in fluid delivery applications). According to some embodiments, cleaning unit 300 may be activated on a regular basis (e.g. for five minutes once a day), either manually or automatically, to ensure that apertures 218 do not become blocked by cell growth.

Making reference again to FIG. 2A, according to some embodiments, power supply unit 30 includes a power supply component 602. Power supply component 602 is electrically coupled/connected to electrical line 410, as elaborated on below. According to some embodiments, and as depicted in FIG. 2A, power supply component 602 is or includes a flat coil 610 of conducting wire mounted on a PCB 604. Flexible extension 40 extends from a proximal end 612 thereof to a distal end 614 thereof. Proximal end 612 is connected to power supply component 602. Distal end 614 may be connected to catheter tube 100, such as to form a Y-junction 620 therewith. Electrical line 410 extends from catheter tip member 200 to power supply unit 30 via (a distal section of) catheter tube 100 and via flexible extension 40.

According to some embodiments, wherein power supply component 602 includes coil 610, vibration generator 400 may be activated by inducing an oscillating magnetic field through coil 610, such as to induce an alternating current via coil 610 and electrical line 410. The alternating current induces an oscillating magnetic field through coil 402, which in turn induces mechanical oscillations of metallic casing 404 and cleaning unit 300. According to some embodiments, wherein power supply unit 30 and flexible extension 40 are implantable, an external activation unit (e.g. a headset (not shown)) may be provided; the external activation unit being configured to generate an oscillating magnetic field, so that, when operated, e.g. by a patient or a caregiver, the generated magnetic field induces an alternating current via coil 610.

According to some embodiments, power supply component 602 may be or include a battery. According to some embodiments, the battery may be rechargeable via wireless power transfer (e.g. using coil 610 or a coil similar thereto, or some other type of receiver).

According to some embodiments, catheter tip member 200 may include a sensor (not shown) configured to monitor the operation of cleaning unit 300. According to such some embodiments, the sensor is or includes a motion sensor configured to monitor movement of cleaning unit 300 when activated. In such embodiments, electrical lines 410 may include additional conductive tracks to relay the signal obtained by the sensor to a processor, which may be housed in power supply unit 30. The processor may be configured to analyze the obtained signal to verify proper operation of cleaning unit 300.

PCB 604 may include electronic circuitry (including, for example, electrical switches, processing circuitry including one or more processors and memory components, etc.) configured to control cleaning unit 300 operation. e.g. to switch on/off cleaning unit 300, to electrically couple/decouple vibration generator 400 and power supply component 602. According to some embodiments, PCB 604 may include a communication unit (e.g. a Bluetooth or RF antenna) configured to communicatively associate PCB 604 with an external controller (such as a mobile communication device) and/or an external activation unit (such as the headset described above), thereby allowing to relay the sensor readings to the mobile communication device/external activation unit. According to some embodiments, power supply component 602 may further be used for communicating with the external activation unit.

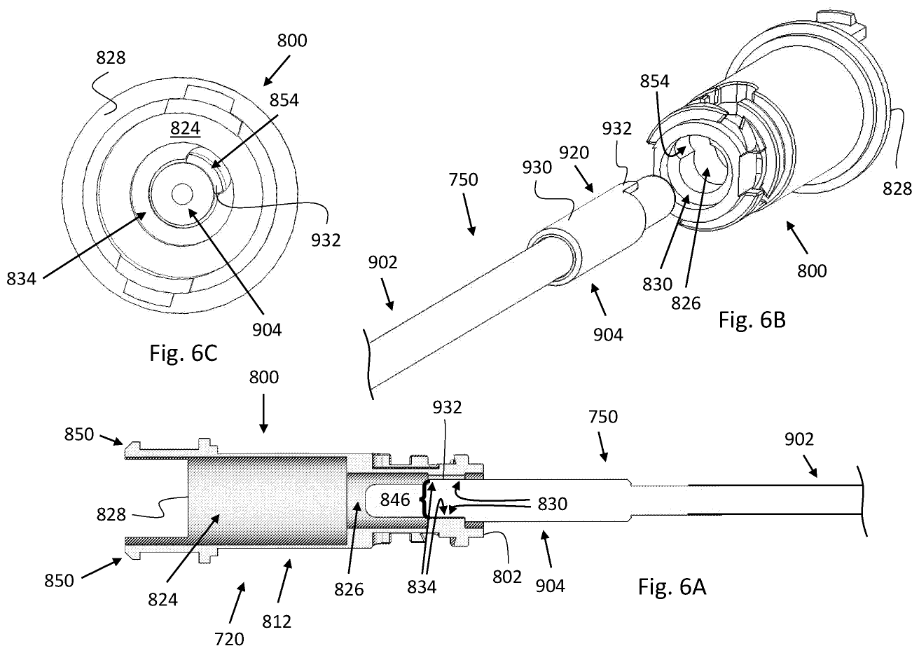

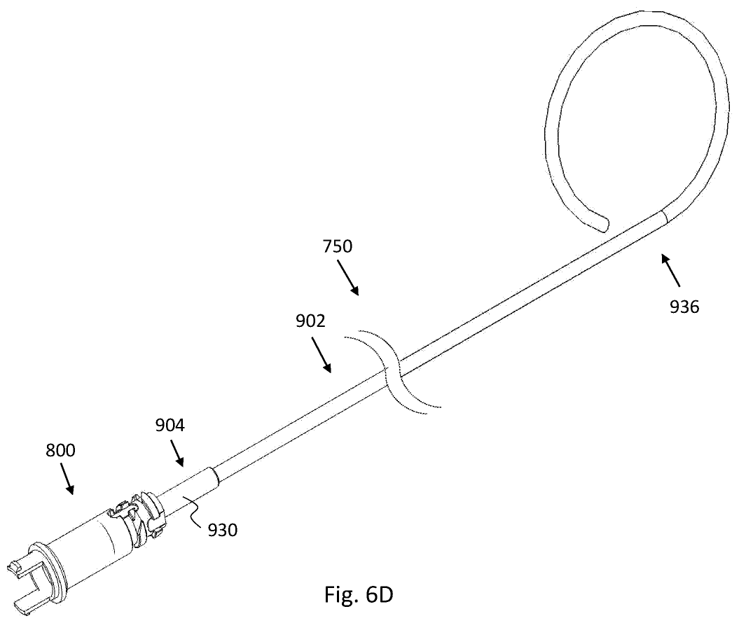

Making reference to FIGS. 6A-6D, FIG. 6A is a schematic, side, cross-sectional view of a catheter tip member (more precisely, the proximal section thereof) of a ventricular catheter 720 and a mandrel 750, which is shown engaging the catheter tip member, according to some embodiments. FIG. 6B is a schematic, perspective, exploded view of the proximal section of the catheter tip member and mandrel 750. FIG. 6C is a schematic view of the proximal section of the catheter tip member and mandrel 750, taken facing the distal end of the proximal section. FIG. 6D is a schematic, perspective view of the proximal section of the catheter tip member and mandrel 750.

Catheter 720 and mandrel 750 are similar to catheter 20 and mandrel 50 but differ therefrom in that when mandrel 750 engages catheter 720, catheter 720 is not free to rotate about the longitudinal axis thereof (which runs parallel to the y-axis) independently of mandrel 750. That is, catheter 720 cannot be rotated unless rotated by, or together with, mandrel 750, as explained below.

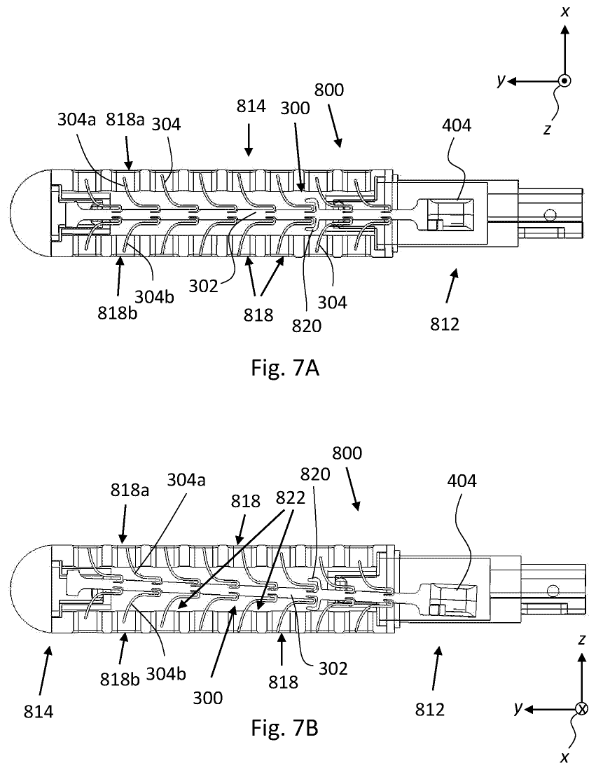

Catheter 720 includes catheter tube 100, a catheter tip member 800, and cleaning unit 300 (shown in FIGS. 7A and 7B). Catheter tip member 800 is similar to catheter tip 212 but differs therefrom at least in including a first key pattern. Mandrel 750 is similar to mandrel 50 but differs therefrom at least in including a second key pattern. The two keys patterns are complementary in the sense that engaging of catheter tip member 800 by mandrel 750 causes catheter tip member 800 and mandrel 750 to interlock such that a rotation of mandrel 750 induces an (equal) rotation of catheter tip member 800, and free rotation of catheter tip member 800 (relative to mandrel 750) is arrested/inhibited.

The interlocking of catheter tip member 800 and mandrel 750 allows a surgeon to controllably orient catheter tip member 800 in its designated location (target site) within the body, e.g. in a brain ventricle, during implantation of catheter 720, as further elaborated on below. In particular, the interlocking allows a surgeon to controllably rotate catheter tip member 800 around the longitudinal axis thereof during the implantation. Thus, the surgeon can orient catheter tip member 800 such that when the subject is standing or sitting upright, pairs of opposite apertures (e.g. apertures 818a and 818b from apertures 818) on the walls of catheter tip member 800 are level (at the same height), so that, according to some embodiments, cleaning unit 300 may be suspended from apertures 818 with the two sets of arms of cleaning unit 300 equally or substantially equally supported by apertures 818 (i.e. all of arms 304 are horizontally disposed on a plane parallel to the xy-plane). This allows a subject or a caregiver to activate cleaning unit 300 when arms 304 are substantially horizontally disposed, so that the motion of tips 316 into and/or out of apertures 818 does not have to overcome gravity (the motion being horizontal or substantially horizontal, since there is no difference in height between the two rows of apertures, as is the case in FIG. 7A, and unlike the case depicted in FIG. 7B). By orienting catheter tip member 800 such that pairs of opposite apertures (e.g. apertures 818a and 818b from apertures 818) on the walls of catheter tip member 800 are level (disposed horizontally), the cleaning of apertures 800 may be improved as compared to when the two sets of arms are not level.

FIG. 7A depicts catheter tip member 800 disposed, such as to that pairs of opposite arms from arms 304, such as arms 304a and 304b are level (essentially parallel to the xy-plane/ground) and equally supported (by apertures 818a and 818b, respectively). In both FIG. 7A and FIG. 7B, the z-axes of the respective depicted coordinate systems point perpendicularly to the ground. In FIG. 7B catheter tip member 800 is shown disposed with one set of arms located above the other (e.g. arm 304a is located above arm 304b), essentially parallel to the yz-plane. That is, in FIG. 7B catheter tip member 800 is rotated by 90° about the longitudinal axis of catheter tip member 800 relative to the orientation thereof in FIG. 7A.

According to some embodiments, cleaning unit 300 further comprises a member 820, projecting radially from shaft 302, and which may be shaped similarly to a trident or a pitchfork. Member 820 may provide a pivot point for cleaning unit 300 tilting motion, being wider than any other component of the cleaning unit 300. When catheter tip member 800 is oriented as depicted in FIG. 7B, member 820 will “rest” on an (inner) bottom wall 822 of catheter tip member 800, so that instead of all of the arms from the bottom set of arms of cleaning unit 300 (equally) falling onto bottom wall 822, only some of the arms from the bottom set of arms will end up resting against bottom wall 822. In particular, since the weights of proximal and distal portions of cleaning unit 300 (i.e. proximally to member 820 and distally thereto) are not equal, cleaning unit 300 will end up tilted, with member 820 serving as the tilting point. In FIG. 7B cleaning unit 300 is tilted clockwise as the torque applied by the proximal portion of cleaning unit 300 including metallic casing 404 is greater than the torque applied by the distal portion, but the opposite option may apply, depending e.g. on the thickness of shaft 302, the materials the various components are made of, and the location of the pivot point.

The orientation depicted in FIG. 7B may be unfavorable as compared to the one depicted in FIG. 7A (per the activation regime wherein the subject is standing or sitting upright), since, when cleaning unit 300 is activated (while oriented as depicted in FIG. 7B), the cleaning action has to overcome gravity (since the motion is on a plane perpendicular to the ground), with the result that proximally located apertures on the top wall of catheter tip member 800 which are above the downwardly tilted end of the cleaning unit 300, and distally located apertures on bottom wall 822, which are below the upwardly tilted end of cleaning unit 300, are potentially not cleaned as effectively as proximally located apertures on bottom wall 822 and distally located apertures on the top wall of catheter tip member 800 (e.g. aperture 818b is potentially not cleaned as well as aperture 818a).

It will be understood that catheter tip member 800 will be oriented differently during implantation if the cleaning action is intended to take place when the subject is not standing or sitting upright. For example, if the cleaning action is intended to take place when the subject is lying down on their side, then during implantation the surgeon will orient catheter tip member 800 accordingly (i.e. such that the two sets of arms are level when the subject is lying down on their side (but not e.g. when the subject is standing)).

More specifically, according to some embodiments, catheter tip member 800 includes a tip member proximal section 812 (i.e. a proximal section of catheter tip member 800) and a tip member distal section 814 (i.e. a distal section of catheter tip member 800), and mandrel 750 includes a mandrel main body 902 (similar to mandrel main body 502) and a mandrel tip portion 904 (a distal portion of mandrel 750). Tip member proximal section 812 includes a stopper 830 in the form of a first geometrical feature 834, which includes the first key pattern. A distal end 828 of tip member proximal section 812 is indicated in FIGS. 6A-6C, and may include snaps 850 (indicated in FIG. 6A) when tip member proximal section 812 and tip member distal section 814 are configured to be connected by a snap-fit mechanism. Mandrel tip portion 904 includes a second geometrical feature 920, which includes the second key pattern.

According to some non-limiting examples, and as depicted in FIG. 6A, first geometrical feature 834 constitutes a narrowed segment 846 of a lumen 826. Similarly, to lumen 226 of catheter tip member 200, lumen 826 distally extends from a tip member proximal end 802 (i.e. a proximal end of catheter tip member 800) to a chamber 824 within tip member proximal section 812. Lumen 826 is configured to receive mandrel tip portion 904. The first key pattern may be in the form of a slot 854 (depression or groove) in narrowed segment 846. Second geometrical feature 920 may be in the form of a band 930 disposed around mandrel main body 902 (e.g. a stiff wire or thin elongated shaft). The second key pattern may be in the form of a tooth 932 extending in the distal direction from the distal edge (not numbered) of band 930. Tooth 932 is configured to be fitted/slotted into slot 854, thereby providing the interlocking of mandrel 750 and catheter tip member 800.

According to some embodiments, the first key pattern may include two or more slots/notches/recesses and the second key pattern may include an equal number of corresponding protrusions/projections (e.g. teeth), such as to allow the interlocking of mandrel 750 and catheter tip member 800. According to some embodiments, the first key pattern may include one or more protrusions/projections (e.g. teeth) projecting from a proximal end (not numbered) of narrowed segment 846 and the second key pattern may include the same number of slots/notches/recesses, such as to allow the interlocking of mandrel tip portion 904 and catheter tip member 800.

According to some embodiments, when mandrel 750 is inserted into catheter 720 such that mandrel 750 engages catheter tip member 800, a mandrel proximal portion 936 (a proximal portion of mandrel 750 shown in FIG. 6D) proximally extends beyond tube proximal end 102. That is, when mandrel 750 is inserted into catheter 720 such that mandrel tip portion 904 engages tip member proximal section 812, mandrel proximal portion 936 is exposed. According to some embodiments, mandrel 750 has a sufficiently high torsional stiffness such that by rotating mandrel proximal portion 936 about the longitudinal axis of mandrel 750, mandrel tip portion 904 is also rotated and to the same, or substantially the same, extent. In particular, mandrel 750 may have sufficiently high torsional stiffness such that mandrel 750 does not twist, or substantially does not twist, when mandrel proximal portion 936 is turned (rotated) about the longitudinal axis of mandrel 750, even when mandrel tip portion 904 is interlocked with catheter tip member 800.

According to some embodiments, and as depicted in FIG. 6D, mandrel proximal portion 936 may be coiled or curled. The coiling provides leverage to facilitate rotating mandrel 750 about the longitudinal axis thereof when mandrel 750 is inserted into catheter 720, which is in turn inserted into a ventricle. It is noted that the coiling of mandrel proximal portion 936 also provides visual indication of the orientation of mandrel 750 within a ventricle, and, as such, allows a surgeon to determine and control the orientation of catheter tip member 800 within a ventricle. More specifically, since the angular relation between the coiling of mandrel proximal portion 936 and tooth 932 is known, and since the angular relation between slot 854 and apertures 818 is also known, a surgeon may controllably orient catheter tip member 800 during implantation thereof using the coiling of mandrel proximal portion 936.

Additionally or alternatively, mandrel proximal portion 936 may be non-symmetrically notched, marked, or colored, so as to provide a visual indication of the orientation of catheter tip member 800. According to some embodiments, mandrel proximal portion 936 may have a non-circular cross-section, thereby providing a visual indication of the orientation of catheter tip member 800.

Although cerebral shunts are perhaps the most commonly used shunts, the skilled person will understand that such shunts, or similar thereto, can be applied to other parts of the body where the drainage of excess fluid is required, such as in urethral catheters, vesicostomy, peritoneal dialysis, and the like. Further, such shunts can also be used in industrial applications where it may be necessary to drain fluids from a remote inaccessible location. The skilled person will also understand that the scope of the disclosure is not limited to drainage catheters and shunts, but more generally covers catheters, shunts, delivery ports, and the like, used in, or additionally in, fluid delivery and passage, such as in the delivery of medication. In particular, catheters such as ventricular catheters 20 and 720 and mandrels 50 and 750, or similar thereto, may be implanted in other body cavities for drainage, delivery, and/or passage of bodily fluids and/or administered fluids. Similarly, assemblies similar to ventricular catheter assembly 10 may be used in such applications and may additionally include pumps, and the like, when the delivery of fluids is, or also is, required.

Even more generally, the skilled person will understand that the scope of the disclosure covers implants, which include sensitive internal components and which include a stopper (such as the stoppers disclosed herein), and the guiding of the implants to target sites in the body (as well as the orienting thereof in the target site) using a mandrel (such as the mandrels disclosed herein).

According to an aspect of some embodiments, there is provided a catheter assembly similar to the catheter assemblies described above (e.g. catheter assembly 10 and catheter assemblies similar thereto but including, in place of catheter 20, catheter 720, or a catheter similar to catheter 720). In addition to being configured to be implanted using a mandrel, such as the above disclosed mandrels (e.g. mandrels 50 and 750), the catheter assembly is configured for monitoring/measuring one or more physical parameters indicative of a condition of a subject implanted with the catheter assembly and/or (proper) functionality of the catheter assembly. Physical parameters indicative of the condition of the subject may include pressure (e.g. intracranial pressure when the catheter assembly is implanted in the brain), temperature, and level of acidity. Physical parameters indicative of functionality of the catheter assembly may include a fluid flow rate through the catheter of the catheter assembly and pressure/temperature therein. The monitoring may be performed essentially continuously (when the catheter assembly includes a power source, such as an implantable battery) or each time a cleaning session is initiated, for example, at least once a day (e.g. when the catheter assembly includes a power supply unit, such as power supply unit 30).

According to some embodiments, a processing circuitry of the catheter assembly (such as the processing circuitry of PCB 604) may be configured to receive measurement data of the one or more physical parameters and to analyze the measurement data. Exceeding predetermined thresholds and/or sharp changes in the measured values of the physical parameters may indicate that medical intervention is required. Trend analysis of the measured values may advantageously allow one to predict in advance the development of a physical condition (which may require medical attention).

According to some such embodiments, the catheter assembly further includes a sensor(s), which is implantable (e.g. housed in the catheter) and configured to monitor the physical parameters. According to some embodiments, the sensor(s) includes a pressure sensor configured to measure the pressure within the catheter and/or the body cavity wherein the catheter is implanted. According to some embodiments, the sensor(s) includes a flow meter configured to measure the fluid-flow rate (or, more generally, fluid flow related parameters) in the catheter.

According to some embodiments, the catheter assembly is further configured for self-activation (i.e. to initiate a cleaning session) on receipt of a signal indicative of occlusion (blockage) in the catheter system (so that the catheter assembly may operate in a closed-loop manner). According to some embodiments, the analysis of the measurement data may be partially or fully performed by external processing circuitry (e.g. included in an external activation unit such as the headset described above), the catheter assembly's processing circuitry being configured to forward the measurement data to the external processing circuitry.

According to an aspect of some embodiments, there is provided a catheter assembly similar to the catheter assemblies described above (e.g. catheter assembly 10 and catheter assemblies similar thereto but including, in place of catheter 20, catheter 720, or a catheter similar to catheter 720), with self-monitoring capabilities. More specifically, in addition to being configured to be implanted using a mandrel, such as the above disclosed mandrels (e.g. mandrels 50 and 750), according to some embodiments, the catheter assembly further includes a (motion) sensor configured to detect motion of a cleaning unit of the catheter assembly (such as cleaning unit 300) and to output a signal(s) indicative of the motion to a processing circuitry of the catheter assembly, such as the processing circuitry of PCB 604. The catheter assembly's processing circuitry may be configured to analyze (process) the signal(s) to determine whether the cleaning unit is working properly (i.e. not malfunctioning), and/or the catheter assembly's processing circuitry may be configured to forward the signal(s) to an external processing circuitry (e.g. included in an external activation unit such as the headset described above, or in an external controller such as a mobile communication device described above), the external processing circuitry being configured to analyze the signal(s) to determine whether the cleaning unit is working properly.

In particular, in embodiments wherein the motion of the cleaning unit is reciprocal/oscillatory, the signal(s) may be processed to compute the amplitude of the motion of the cleaning unit and/or the mean (average) position of the cleaning unit: A small amplitude may be indicative of limited motion due to blockage, e.g. of one or more of the apertures in a catheter tip member (such as catheter tip member 200 or catheter tip member 800) of the catheter, and/or a malfunction in the cleaning unit (or in other components associated thereto). A mean position which is displaced relative to the “normal” mean position (that is the mean position when the cleaning unit is working properly) may be indicative of unilateral blockage or partial blockage. According to some embodiments, if the analysis of the signal(s) indicates a malfunction, a corrective action may be initiated to rectify the malfunction. The corrective action may include increasing a power supplied to the cleaning unit, changing a duty cycle of the cleaning unit, and/or changing an activation waveform of the cleaning unit.

According to some such embodiments, the sensor may be an optical sensor and/or a proximity sensor. According to some embodiments, wherein a vibration generator (such as vibration generator 400) of the catheter assembly includes an electromagnet, the sensor may be a magnetic sensor (e.g. a Hall effect sensor) configured to detect motion of a metallic casing including/housing a magnet of the electromagnet. In such embodiments, the sensor may be positioned in proximity to the metallic casing, e.g. at, or near, a distal end of the catheter tube (the catheter tube being connected at the distal end thereof to the catheter tip member).

As used herein, according to some embodiments, the term “pattern” may refer to a spatial structural on a surface or edge of an object/element, such as one or more grooves and/or projections on the surface.

As used herein, according to some embodiments, the term “torsional stiffness” with reference to an elongated object (e.g. element), refers to the object's resistance to twisting about the longitudinal axis of the object.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purposes and should not be regarded as limiting. Citation or identification of any reference in this application shall not be construed as an admission that such reference is available as prior art to the disclosure. Section headings are used herein to ease understanding of the specification and should not be construed as necessarily limiting.

1-30. (canceled)

31. An implantable catheter for fluid passage, comprising:
a catheter tube;
an elongated and hollow catheter tip member comprising one or more apertures and an open proximal end, the catheter tip member being connected at the proximal end thereof to the catheter tube, such as to fluidly couple the catheter tube to an exterior of the catheter; and
a cleaning unit at least partially positioned within the catheter tip member;
wherein the cleaning unit is configured for movement within the catheter tip member such as to mechanically prevent, mitigate, and/or remove blockage of at least one of the one or more apertures when the catheter tip member is implanted in a body cavity; and
wherein the catheter tip member further comprises a stopper configured to be engaged by a tip portion of a mandrel, such as to prevent the mandrel from at least one of reaching and damaging the cleaning unit when using the mandrel to guide the catheter tip member into the body cavity.

32. The implantable catheter of claim 31, wherein the catheter is a ventricular catheter for draining fluids, wherein the fluids comprise cerebrospinal fluid (CSF), and wherein the body cavity comprises a brain ventricle.

33. The implantable catheter of claim 31, wherein the catheter tip member comprises a tip member proximal section, comprising the stopper, and a tip member distal section, comprising the one or more apertures, and wherein the cleaning unit is at least partially housed within the tip member distal section.

34. The implantable catheter of claim 33, wherein the stopper comprises a first geometrical feature projecting from an inner surface of the tip member proximal section.

35. The implantable catheter of claim 34, wherein the first geometrical feature comprises one or more of: a flange extending along a circumference of the inner surface, a narrowed segment of a lumen defined by the inner surface, and at least two spaced-apart ridges along the circumference of the inner surface.

36. The implantable catheter of claim 31, wherein the cleaning unit comprises an elongated shaft comprising one or more arms configured to project into the one or more apertures and to move therein, and
wherein the cleaning unit is configured to allow vibration thereof and wherein the movement of the one or more arms within the one or more apertures is induced by the vibration of the cleaning unit.

37. The implantable catheter of claim 36, wherein the one or more arms of the cleaning unit extend into the one or more apertures such as to suspend the cleaning unit within the catheter tip member.

38. The implantable catheter of claim 31, further comprising a vibration generator configured to induce the movement of the cleaning unit, and wherein the vibration generator is connected to an electrical line which is configured to couple to a power supply unit for powering the vibration generator.

39. The implantable catheter of claim 38, wherein the catheter tube comprises a port, wherein the electrical line extends into the port, and wherein the port is configured to electrically couple to the power supply unit.

40. The implantable catheter of claim 38, wherein the cleaning unit comprises a metallic member comprising at least one of a magnetic material and magnetizable material, and wherein the tip member further comprises an electrically conducting coil, such that the metallic member and the electrically conducting coil are configured as an electromagnet constituting, or included in, the vibration generator.

41. A kit for fluid passage in a body cavity, the kit comprising:
an implantable catheter comprising:
a catheter tube;
an elongated and hollow catheter tip member comprising one or more apertures, an open proximal end and a stopper, the catheter tip member being configured for connection at the open proximal end thereof to the catheter tube, such as to fluidly couple the catheter tube to an exterior of the catheter; and
a cleaning unit positioned within the catheter tip member, the cleaning unit being configured for movement within the catheter tip member such as to mechanically prevent, mitigate, and/or remove blockage of at least one of the one or more apertures when the catheter tip member is implanted in a body cavity, and
a mandrel comprising a proximal portion and a tip portion, the tip portion being configured to engage the stopper of the catheter tip member, such as to prevent the mandrel from at least one of reaching and damaging the cleaning unit when using the mandrel to guide the catheter tip member into the body cavity.

42. The kit of claim 41, wherein the mandrel is pre-installed in the implantable catheter such that the tip portion of the mandrel engages the stopper of the catheter tip member.

43. The kit of claim 41, wherein the stopper comprises a first geometrical feature projecting from an inner surface of the tip member proximal section, and wherein the tip portion of the mandrel comprises a second geometrical feature projecting from an outer surface of the tip portion of the mandrel, the second geometrical feature being configured to engage the first geometrical feature.

44. The kit of claim 43, wherein the first geometrical feature comprises one or more of: a flange extending along a circumference of the inner surface, a narrowed segment of a lumen defined by the inner surface, and at least two spaced-apart ridges along the circumference of the inner surface, and the second geometrical feature comprises a band or a flange extending along a circumference of the outer surface of the tip portion of the mandrel.

45. The kit of claim 41, wherein the stopper comprises a first key pattern and the tip portion of the mandrel comprises a second key pattern complementary to the first key pattern, the first and second key patterns being configured to interlock upon engaging of the stopper by the tip portion of the mandrel, such that a rotation of the mandrel induces an equal rotation of the catheter tip member;
wherein a proximal portion of the mandrel proximally extends outside the catheter tube; and
wherein the mandrel comprises an orientation indicator.

46. The kit of claim 45, wherein the proximal portion of the mandrel is coiled and constitutes at least part of the orientation indicator.

47. The kit of claim 45, wherein one of the first and second key patterns is configured as male and the other of the first and second key patterns is configured as female.

48. The kit of claim 45, wherein the stopper comprises a first geometrical feature projecting from an inner surface of the tip member proximal section, and the tip portion of the mandrel comprises a second geometrical feature projecting from an outer surface of the tip portion of the mandrel, and wherein at least one of:
(i) the first key pattern comprises at least one slot in the first geometrical feature and the second key pattern comprises at least one projection extending distally from a distal end of the second geometrical feature, the at least one slot being complementary to the at least one projection, and
(ii) the first key pattern comprises at least one projection extending proximally from a proximal end of first geometrical feature and the second key pattern comprises at least one slot in the second geometrical feature, the at least one slot being complementary to the at least one projection.

49. The kit of claim 41, wherein a proximal section of the catheter tube is attached to the proximal portion of the mandrel, thereby facilitating maintaining the engaging of the stopper by the tip portion of the mandrel when guiding the catheter tip member into the body cavity.

50. The kit of claim 49, wherein the proximal section of the catheter tube is detachable from the rest of the catheter tube, thereby facilitating removing the mandrel from the implantable catheter.
